(19) United States
(12) Patent Application Publication
(10) Pub. No.: US 2017/0072049 A1
(43) Pub. Date: Mar. 16, 2017

(54) A Composition Comprising Ex-Vivo Generated Dendritic Cells

(71) Applicant: Shakti Upadhyay, Mumbai (IN)

(72) Inventor: Shakti Upadhyay, Mumbai (IN)

(21) Appl. No.: 15/122,718

(22) PCT Filed: Mar. 5, 2015
(86) PCT No.: PCT/IN2015/050017

§ 371 Date: Aug. 31, 2016

(30) Foreign Application Priority Data
Mar. 4, 2014 (IN) 2876/MUM/2013

(51) Int. Cl. A61K 39/39; A61K 39/00

(52) U.S. Cl. CPC A61K 39/39; A61K 39/0011; A61K 2039/5154; A61K 2039/55583

(57) ABSTRACT

The present invention relates to a composition comprising ex-vivo generated antigen-loaded dendritic cells in combination with β-glucan and hyaluronic acid or pharmaceutically acceptable derivatives thereof and methods of preparing and delivering the same. The present composition of the present invention enhances antigen-specific T cell response against cancer cells. The composition or formulation comprising the same is delivered through injection, biocampitable scaffold or implant by intradermal, subcutaneous, intramuscular, intratumoral, or intranodal administration for providing an effective immune response for treatment and/or prevention of cancer progression, recurrence, and/or metastasis in a cancer patient.

### FIELD OF THE INVENTION

The present invention relates to an ex-vivo generated antigen-loaded dendritic cells based composition and methods of preparing and delivering the same. More particularly, the present invention relates to a composition for enhancing antigen-specific T cell response against cancer cells. Even more specifically the present invention relates to an ex-vivo generated antigen-loaded dendritic cells based composition and methods of preparing and delivering the same for enhancing antigen-specific T cell response for providing an effective immune response for treatment and/or prevention of cancer progression, recurrence, and/or metastasis in a cancer patient more specifically by intradermal or subcutaneous delivery.

### BACKGROUND OF THE INVENTION

Dendritic Cell (DC) based immunotherapy is a strategy for educating or activating the immune system, body's defense forces, to react against and kill the cancer cells or pathogens. The advantage of this therapeutic strategy is that the immune system is selective and attacks only abnormal (cancer) or foreign cells (pathogens) ignoring the normal healthy ones. This is in contrast to chemotherapy that uses drugs, which directly act upon the cancer cells and pathogens and often kill normal cells as well; chemotherapy consequently is associated with undesirable side effects that severely compromise the quality of life. Surgery, on the other hand, is possible only for localized solid tumors and such surgical intervention has its own risk and limitations.

Generation of immune response however depends on the ability of the DCs to identify diseased (abnormal/infected) cells and to process the captured antigens for presentation to T cells (helper), which in turn generate antigen-specific Cytotoxic T cells or activate antigen-specific B cells to produce neutralizing antibodies, to eliminate the diseased cell or pathogens. This process also generates memory cells that can quickly launch immune attack during any future recurrence or encounters with the same antigens.

Much effort has been made over the last decade to use DCs as therapeutic vaccines to induce effective anticancer immune responses. However, the great hope provided by these investigations has not translated in terms of clinical efficacy. Thus one of the challenges resides in optimizing DC-based therapy to give maximum clinical efficacy.

Cell-mediated immunity plays an important role in immune responses against cancer. CD8+ cytotoxic T lymphocytes (CTLs) are key effector cells in anticancer immunity. It has however been shown that in patients with advanced cancers, circulating DCs and those residing in the proximity of the cancer tissue or cells are not very functional. In particular, their capacity to take up and process antigens or to migrate upon maturation is often severely altered.

Consequently, the most important factors that account for the poor anticancer responses is the lack of CD4+ T-cell help. CD4+ T helper cells play an important role in the development of effective anticancer immunity. Both the number and function of cancer-specific CTL are significantly enhanced in the presence of cancer-specific CD4+ T-cell responses, whereas depletion or silencing of CD4+ T cells facilitates cancer progression and abrogates the survival of cancer-bearing hosts, indicating the importance of cancer-specific CD4+ T-cell help in maintaining the cancer-reactive CTL function in vivo. Therefore, CD4+ T-cell help is critical for promoting effective anticancer CTL responses, which is achieved not only by maintaining the numbers of cancer-specific CD8+ T cells but also by the optimal CTL function. Recent studies have shown that a specific subset of CD4+ T− helper cells, TH17, significantly contributes to anti-cancer immunity.

The most important aspect of an effective immune response is the maturation and interaction of DCs with T lymphocytes. While DCs have been generated ex-vivo and various protocols have been attempted for antigen loading and maturation, the results have been variable and not very successful. Although phenotypic analysis of DCs following ex-vivo maturation in culture medium may demonstrate requisite surface markers, functionally the DCs may fail to drive the immune response in effective manner following transfer into the host.

Thus, there is a need for arriving at a dendritic cells based composition and a method of delivering the same for enhancing antigen-specific T cell response that is to say for providing effective immune response for treatment and/or prevention of cancer progression, recurrence, and/or metastasis in a cancer patient.

### SUMMARY

Accordingly, the present invention in one aspect provides a composition comprising ex-vivo generated antigen-loaded dendritic cells in combination with β-glucan and hyaluronic acid or pharmaceutically acceptable derivatives thereof.

In one embodiment the present invention provides a composition comprising ex-vivo generated antigen-loaded dendritic cells in combination with β-glucan and hyaluronic acid or pharmaceutically acceptable derivatives thereof for intradermal or subcutaneous delivery.

In one embodiment, the present invention provides a composition comprising ex-vivo generated antigen-loaded dendritic cells in combination with β-glucan and hyaluronic acid or pharmaceutically acceptable derivatives thereof for enhancing antigen-specific T cell response.

In one embodiment the present invention provides a composition comprising ex-vivo generated antigen-loaded dendritic cells in combination with β-glucan and hyaluronic acid or pharmaceutically acceptable derivatives thereof for enhancing antigen-specific T cell response against a solid tumor or hematological cancer.

In one embodiment, the present invention provides a composition comprising ex-vivo generated antigen-loaded dendritic cells in combination with β-glucan and hyaluronic acid or pharmaceutically acceptable derivatives thereof for enhancing antigen-specific T cell response for providing effective immune response for treatment and/or prevention of cancer progression, recurrence, and/or metastasis in a cancer patient.

In one embodiment the present invention provides a method for preparing a composition comprising ex-vivo generated antigen-loaded dendritic cells in combination with β-glucan and hyaluronic acid or pharmaceutically acceptable derivatives thereof comprising the steps of: loading ex-vivo generated dendritic cells with an antigen preparation in presence of a mixture of β-glucan and hyaluronic acid or pharmaceutically acceptable derivatives thereof.

In one embodiment the present invention provides the use of a composition comprising ex-vivo generated antigen-loaded dendritic cells in combination with β-glucan and hyaluronic acid or pharmaceutically acceptable derivatives thereof for intradermal or subcutaneous delivery for enhancing an antigen-specific T cell response.

In one embodiment the present invention provides the use of a composition comprising ex-vivo generated antigen-loaded dendritic cells in combination with β-glucan and hyaluronic acid or pharmaceutically acceptable derivatives thereof for intradermal or subcutaneous delivery for enhancing antigen-specific T cell response against a solid tumor or hematological cancer cells.

In one embodiment the present invention provides a method for enhancing antigen-specific T cell response in a cancer patient comprising delivering intradermally or subcutaneously to a cancer patient a composition comprising ex-vivo generated antigen-loaded dendritic cells in combination with β-glucan and hyaluronic acid or pharmaceutically acceptable derivatives thereof.

In one embodiment the present invention provides a method for treatment and/or prevention of cancer progression, recurrence, and/or metastasis in a cancer patient comprising an intradermal or subcutaneous administration of a composition comprising ex-vivo generated antigen-loaded dendritic cells in combination with β-glucan and hyaluronic acid or pharmaceutically acceptable derivatives thereof.

### BRIEF DESCRIPTION OF THE DRAWINGS

Other objects, features, and advantages of the invention will be apparent from the following description when read with reference to the accompanying drawings.

FIG. 1 shows purity of CD14+ monocytes isolated from peripheral blood mononuclear cells (PBMC) to be >95%.

FIG. 2 shows the flowcytometer result evaluating the optimal dose based on phenotypic expression of CD83. FIG. 2-c(iii) demonstrates optimal result of >67% phenotypic expression of CD83 with the combination of GM-CSF (50 ng/ml) and IL-4 (25 ng/ml).

FIG. 3 shows flowcytometric analysis of antigen uptake by dendritic cells, using dextran coated FITC beads added to the composition comprising ex-vivo generated antigen-loaded dendritic cells in combination with β-glucan and sodium hyaluronate (DC-TL-BG-HA).

FIG. 4 shows the phenotypic analysis of DCs in DC-TL-BG-HA composition by expression of the surface markers CD14, CD40, CD80, CD86, HLD DR & CD83.

FIG. 5 is a flowchart of an ex-vivo experimental model protocol for evaluating functional efficacy of the compositions comprising DC-TL-HA+CD8; DC-TL-BG+CD8 and DC-TL-BG-HA.

FIG. 6 shows flow cytometry results comparing cell killing determined by CFSE+/7AAD+ staining of various experimental groups: FIG. 6a: CD8 (control group); FIG. 6b: DC+CD8; FIG. 6c: DC-TL+CD8; FIG. 6d: DC-TL-HA+CD8; FIG. 6e: DC-TL-BG+CD8; and FIG. 6f: DC-TL-BG-HA+CD8.

FIG. 7 represents result of administration of the composition DC-TL-BG-HA to a chronic Lymphocytic leukemia patient showing increase in the number of CD8+ T lymphocytes and enhanced production of g-IFN indicating effective tumor antigen sensitization.

### DETAILED DESCRIPTION OF THE INVENTION

The present invention provides a composition comprising ex-vivo generated antigen-loaded dendritic cells in combination with β-glucan and hyaluronic acid or pharmaceutically acceptable derivatives thereof.

In one of the embodiment the present invention provides a composition comprising ex-vivo generated, antigen-loaded autologus DCs, β-glucan and hyaluronic acid or pharmaceutically acceptable derivatives thereof.

In one embodiment the present invention provides a composition comprising ex-vivo generated antigen-loaded dendritic cells in combination with β-glucan and hyaluronic acid or pharmaceutically acceptable derivatives thereof for intradermal or subcutaneous delivery.

In one embodiment, the present invention provides a composition comprising ex-vivo generated antigen-loaded dendritic cells in combination with β-glucan and hyaluronic acid or pharmaceutically acceptable derivatives thereof for enhancing antigen-specific T cell response.

In one embodiment the present invention provides the composition comprising ex-vivo generated antigen-loaded dendritic cells in combination with β-glucan and hyaluronic acid or pharmaceutically acceptable derivatives thereof for enhancing antigen-specific T cell response against a solid tumor or hematological cancer.

In one embodiment, the present invention provides a composition comprising ex-vivo generated antigen-loaded dendritic cells in combination with β-glucan and hyaluronic acid or pharmaceutically acceptable derivatives thereof for enhancing antigen-specific T cell response for providing effective immune response for treatment and/or prevention of cancer progression, recurrence, and/or metastasis in a cancer patient.

In one embodiment the ex-vivo that is in-vitro generated dendritic cells are autologus.

In one of the embodiment, the composition comprises ex-vivo generated autologus dendritic cells ranging from 1×10⁴-1×10¹⁰.

In one embodiment the autologus ex-vivo generated dendritic cells are loaded with antigen.

In one of the preferred embodiment, the antigen is a cancer antigen.

In one embodiment, the antigen is obtained from cancer tissue or cells collected from a cancer patient.

In one embodiment, the antigen is derived from an established cancer cell lines.

In some embodiments, the β-glucan comprised in the composition may be derived from any natural product including, but not limited to, yeast, bacteria or mushrooms.

In some of the embodiments, the β-glucan may be hydrolyzed or micronized oligosaccharide or small 10-20 mer, preferably 10-15 mer polysaccharide or equivalent synthetic oligosaccharide.

In one embodiment, the pharmaceutically acceptable derivative of β-glucan includes any pharmaceutically acceptable salts, solvates, esters or carbamates.

In one embodiment the pharmaceutically acceptable derivative of β-glucan polysaccharide or oligosaccharides is a salt derivative.

In one embodiment, β-glucan or its pharmaceutically acceptable derivative may be linked to a peptide or protein.

In some embodiments the β-glucan to be included in the composition may be β-glucan polysaccharides comprising of at least one of β (1,3), or β (1,6), or β (1,3/1,4), or β (1,3/1,6) glycosidic linkages.

In one of the embodiment, the β-glucan polysaccharide is a linear 1, 3 β-glucan, with or without branching.

In one embodiment, the β-glucan comprised in the composition has the molecular weight of at least 1500 Dalton. In another embodiment, the β-glucan polysaccharide has the molecular weight from 2000-200,000 Dalton. In still further embodiment the β-glucan polysaccharide has the molecular weight from 5,000-50,000 Dalton. In one of the embodiment, the β-glucan polysaccharide has the molecular weight from 10,000-20,000.

The composition of the present invention comprises at least 0.05% of β-glucan on w/v basis.

In one embodiment, the composition comprises β-glucan in the range of 0.1% to 10% w/v basis.

In one embodiment, the composition comprises β-glucan in the range of 1% to 5% on w/v basis.

The hyaluronic acid or its pharmaceutically acceptable derivative comprised in the composition may be in free or cross-linked hydrogel form.

In one embodiment, the pharmaceutically acceptable derivative of hyaluronic acid includes any pharmaceutically acceptable salts, solvates, esters or carbamates.

In one embodiment the pharmaceutically acceptable derivative of hyaluronic acid is a salt derivative.

The composition of the present invention comprises at least 0.01% hyaluronic acid or its pharmaceutically acceptable derivative on w/v basis.

In an embodiment, the composition comprises hyaluronic acid or its pharmaceutically acceptable derivative in the range of 0.1% to 5% on w/v basis.

In one embodiment, the composition comprises hyaluronic acid or its pharmaceutically acceptable derivative in the range of 0.25% to 2.5% on w/v basis.

In one embodiment, the composition comprises hyaluronic acid or its pharmaceutically acceptable derivative in the range of 0.5%-2% on w/v basis.

In some embodiments, the composition as per the present invention may comprise of β-glucan and hyaluronic acid substituted by any physically, chemically or functionally equivalent natural or synthetic entities or product.

In some embodiments, the composition of present invention optionally comprises of additional active component, adjuvant or the like for example the composition may optionally include suitable immunomodulators for examples pattern-recognition receptor ligands or Toll-Like Receptor (TLR) ligands or the like.

In another aspect the present invention provides a method of preparing a composition comprising ex-vivo generated antigen-loaded dendritic cells in combination with β-glucan and hyaluronic acid or pharmaceutically acceptable derivatives thereof wherein, the method comprises loading ex-vivo generated dendritic cells with an antigen preparation in presence of a mixture of β-glucan and hyaluronic acid or pharmaceutically acceptable derivatives thereof.

In some embodiments, the dendritic cells may be autologus dendritic cells generated ex-vivo that is in-vitro from peripheral blood derived CD14+ monocytes or CD34+ hematopoietic stem cells by contacting them with at least one cytokine, immunomodulators, or ligands of pattern recognition receptors.

In some embodiments the ex-vivo generated autologus dendritic cells may be cryopreserved until further use.

The loading of the autologus dendritic cells with antigens is carried out by at least one of the approaches selected from but not limiting to co-culturing dendritic cells with the antigen preparation, electroporation or incorporating antigen preparation in liposome, nano-particles, virus like particles, or any such delivery systems.

In one embodiment an antigen preparation may be selected for prevention/treatment of a specific cancer progression, recurrence, and/or metastasis in a cancer patient

In some of the embodiments, the antigen preparation used for loading the autologus DC is at least one selected from but not limiting to a cancer tissue or cell lysate, a membrane preparation, a partially purified preparation, a substantially purified preparation, as a recombinant expressed protein or portion thereof, a peptide, or proteins expressed on the surface of a recombinant cell, mRNA, or vectors carrying or expressing relevant gene for specific antigenic proteins, or antigen preparation mixed with specific antibody or antibodies against it.

In one embodiment, the antigen preparation is a cancer antigen preparation.

In one embodiment, the antigen preparation is obtained from cancer tissue or cells collected from a cancer patient.

In one embodiment, the antigen preparation is derived from an established cancer cell lines.

For cancer antigen preparation, the cancer tissue or cancer cells or established cancer cell lines are subjected to repeated freeze-thaw cycles, or high-pressure cell disruption or homogenization. Alternatively, the cancer tissue or cells may be exposed to UV and/or elevated temperatures, followed by repeated freeze-thaw cycles, or high-pressure cell disruption or homogenization. Cancer cell lysate thus obtained is subjected to centrifugation, filtration and estimated for total protein content to serve as an antigen preparation.

In some of the preferred embodiments, the cryopreserved dendritic cells can be revived and contacted with the desired antigen preparation for at least 1 hour, preferably for 1-24 hours, washed and re-suspended in a medium comprising of a desired quantities of hyaluronic acid and β-glucan or pharmaceutically acceptable derivatives thereof before administration to the patient.

In alternate preferred embodiments, the cryopreserved dendritic cells can be revived, washed and contacted with the desired antigen preparation for at least 1 hour, preferably for 1-24 hours, washed and re-suspended in a medium comprising of desired quantities of hyaluronic acid and β-glucan or pharmaceutically acceptable derivatives thereof and cryopreserved in aliquots, till further use.

The composition as per the present invention comprising ex-vivo generated antigen-loaded dendritic cells in combination with β-glucan and hyaluronic acid or pharmaceutically acceptable derivatives thereof is used for intradermal or subcutaneous delivery.

The composition as per the present invention may be formulated preferably in the form of a solution or suspension. The formulation comprising composition of the present invention optionally includes pharmaceutically acceptable excipients selected from but not limiting to a suitable buffer, a suitable tonicizing agent and, where appropriate a suitable cryoprotectant.

Examples of buffer includes sodium phosphate, sodium acetate, sodium citrate, or the like which can adjust pH between about 6.0 to 8.0.

Examples of suitable tonicizing agents include glycerol, glucose, mannitol, sodium chloride, calcium or magnesium compounds such as, for example, CaCl2, MgCl2, or the like.

The formulation is delivered through injection, biocampitable scaffold or implant by intradermal, subcutaneous, intra-muscular, intra-tumoral, or intra-nodal administration.

The formulation is delivered to the subject in the need thereof by injecting the appropriate quantity of the formulation intradermally or subcutaneously at multiple sites to a subject in need of such intervention.

In some of the embodiments, the present invention provides a method of delivering the composition, by injecting the therapeutically effective quantity of the composition intradermally, subcutaneously, intra-muscularly, intra-tumorally, or intra-nodally to a subject in need of such intervention at a desired interval.

In some of the embodiments, the present invention provides a method of delivering the composition, by injecting the therapeutically effective quantity of the composition intradermally to a subject in need of such intervention one or more time or as required for enhancing cancer-specific T cell response.

The composition comprising of ex-vivo generated antigen-loaded autologus dendritic cells in combination with β-glucan and hyaluronic acid or pharmaceutically acceptable derivatives thereof or the formulation comprising the composition is delivered intra-dermally, sub-cutaneously, intra-muscularly, intra-tumorally, or intra-nodally to a subject in the need thereof for antigen processing and presentation by the said dendritic cells in situ within patients own tissue environment for enhancing T cell response and generation of an effective immune response for prevention and treatment of any type of cancer.

Cancer means any type of cancer including but not limited to solid tumors and hematological cancers. Solid tumors means an abnormal mass of cells which may stem from different tissue types such as liver, colon, breast, or lung, which initially grows in the organ of its cellular origin and may spread to other organs through metastatic tumor growth in advanced stages of the disease. Hematological cancer means cancer affecting blood, bone marrow, and lymph nodes.

In one embodiment the present invention provides the use of a composition comprising ex-vivo generated antigen-loaded dendritic cells in combination with β-glucan and hyaluronic acid or pharmaceutically acceptable derivatives thereof for intra-dermal, sub-cutaneous, intra-muscular, intra-tumoral, or intra-nodal delivery for enhancing antigen-specific T cell response against a solid tumor or hematological cancer cells.

In one embodiment the present invention provides a method for enhancing antigen-specific T cell response in a cancer patient comprising delivering intra-dermally, sub-cutaneously, intra-muscularly, intra-tumorally, or intra-nodally to a cancer patient a composition comprising ex-vivo generated antigen-loaded dendritic cells in combination with β-glucan and hyaluronic acid or pharmaceutically acceptable derivatives thereof.

The composition can be used alone or in combination with any other therapeutic approaches including, surgery, chemotherapy, radiotherapy and other biological therapy, so as to achieve the best therapeutic or prophylactic effects.

The composition as per the present invention is used for generating an effective antigen-specific T cell response, in the host following intra-dermal, sub-cutaneous, intra-muscular, intra-tumoral, or intra-nodal delivery.

In some embodiments the present invention provides a method for treatment and/or prevention of cancer progression, recurrence, and/or metastasis in a cancer patient by administering to a patient in the need thereof a therapeutically effective amount of a composition comprising of ex-vivo generated antigen-loaded autologus dendritic cells in combination with β-glucan and hyaluronic acid or pharmaceutically acceptable derivatives thereof.

In further aspect the present invention provides a method for treatment and/or prevention of cancer progression, recurrence, and/or metastasis in a cancer patient by delivering intra-dermally, sub-cutaneously, intra-muscularly, intra-tumorally, or intra-nodally to a patient in the need thereof a therapeutically effective amount of a composition comprising of ex-vivo generated antigen-loaded autologus dendritic cells in combination with β-glucan and hyaluronic acid or pharmaceutically acceptable derivatives thereof.

Cancer may be any type of cancer including but not limited to solid tumors and hematological cancers.

In some embodiments the present invention provides a method for treatment and/or prevention of solid tumors in a cancer patient by delivering intra-dermally, sub-cutaneously, intra-muscularly, intra-tumorally, or intra-nodally to a patient in the need thereof a therapeutically effective amount of a composition comprising of ex-vivo generated antigen-loaded autologus dendritic cells in combination with β-glucan and hyaluronic acid or pharmaceutically acceptable derivatives thereof.

Solid tumor may be a cancerous tumor including but not limited to the ones occurring in the patient's prostate, stomach, liver, spleen, pancreas, colon, kidney, gall bladder, ovary, testicle, penis, rectum, lung, trachea, breast, heart, brain, thyroid, parathyroid, pituitary, thymus, muscle, head, neck, skin, retina, uvea, conjunctiva, salivary gland, adrenal gland, throat, esophagus, sweat glands and sebaceous glands.

In some embodiments the present invention provides a method for treatment and/or prevention of hematological cancer in a cancer patient by delivering intra-dermally, sub-cutaneously, intra-muscularly or intra-nodally to a patient in the need thereof a therapeutically effective amount of a composition comprising of ex-vivo generated antigen-loaded autologus dendritic cells in combination with β-glucan and/or Hyaluronic acid or pharmaceutically acceptable derivatives thereof.

Hematological cancer may be a cancer including but not limited to the ones occurring in the patient's blood, bone, marrow, and lymph nodes.

The composition of the present invention is advantageous in the sense that it enhances T cell response for generation of an effective immune response for prevention and treatment of any type of cancer.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention.

The following examples are provided merely as illustrative of various aspects of the present invention and should not be construed to limit the invention in any way.

### EXAMPLES

### Example 1

### Isolation of Monocytes

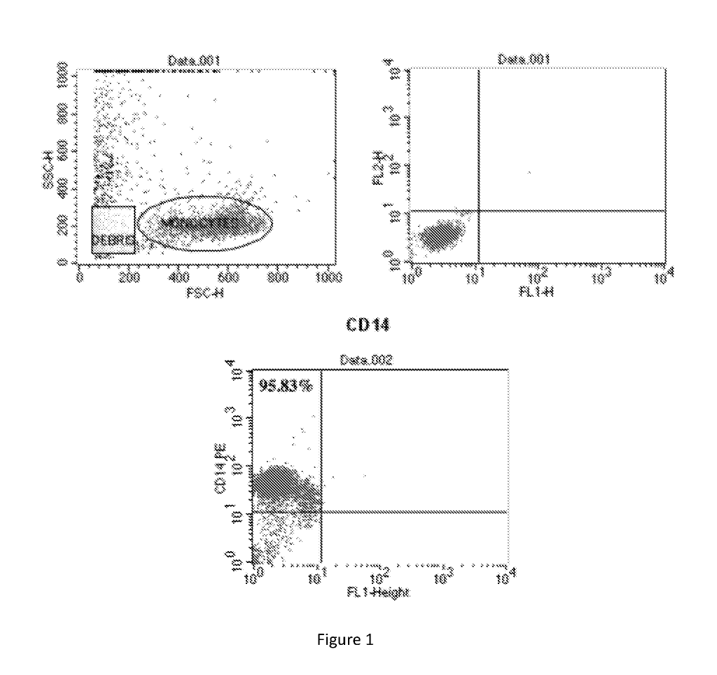

Peripheral blood mononuclear cells (PBMC) were purified using gradient separation and CD14+ monocytes were separated from the total leukocytes by simple adherence to culture plates. FIG. 1 shows purity of CD14+ monocytes isolated from peripheral blood mononuclear cells (PBMC) to be >95%.

### Example 2

### Generation of Dendritic Cells from Monocytes

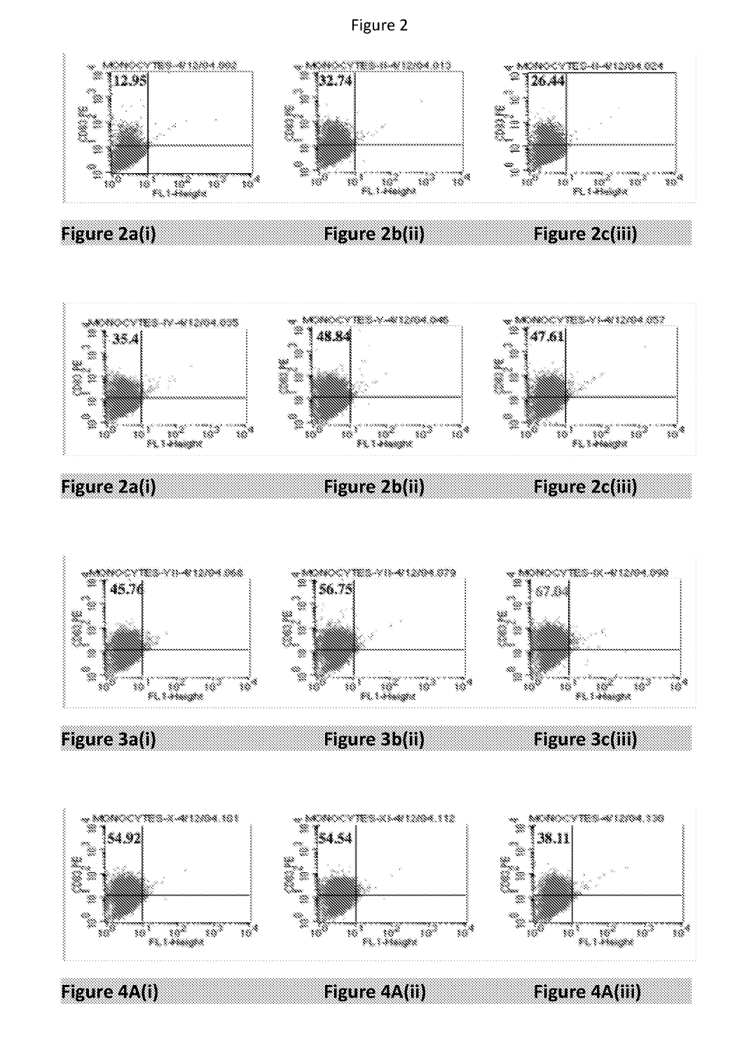

Isolated CD14+ cells were cultured at a concentration of 2×10⁶ cells/ml in sterile culture flasks for 5 days in serum free media at 37° C. in a humidified 5% CO₂ atmosphere in the presence of clinical grade recombinant human (rh) granulocytemacrophage colony-stimulating factor (GM-CSF), 25-250 ng/ml, and rh-IL-4, 25-100 ng/ml. On day 3, half of the medium was replaced with same volume containing fresh rh-GM-CSF and rh-IL-4, at same concentrations. The optimal dose was evaluated based on phenotypic expression of CD83 using a Flowcytometer (FIG. 2). In this experiment, combination of GM-CSF (50 ng/ml) and IL-4 (25 ng/ml) demonstrated the optimal result (FIG. 2-C3).

### Example 3

### Preparation of Tumor Lysate (TL)-β-Glucan (BG)-Hyaluronic Acid (HA) Composition

Tumor tissues obtained following surgery/biopsy were cut into small pieces and then exposed to UV radiation (1-10 mJ/cm²/s) for 10-20 minutes and then kept in the CO₂ incubator maintained at 39-45° C., for 4-8 hours. Following that tissue blocks were lysed by five freeze and thaw cycles, followed by centrifugation at 2000 rpm (738 g) for 10 minutes. Supernatant was removed, filtered and assayed for total protein concentration. Tumor lysate (50-200 ug/ml of protein) was mixed with β-Glucan (50-200 ug/ml) and sodium hyaluronate (0.1-2%) and ultra-sonicated for 1-10 minutes on ice bath. This preparation was aliquoted and cryopreserved in liquid nitrogen tanks till further use.

### Example 4

### Preparation of Dendritic Cell (DC)-TL-BG-HA Composition

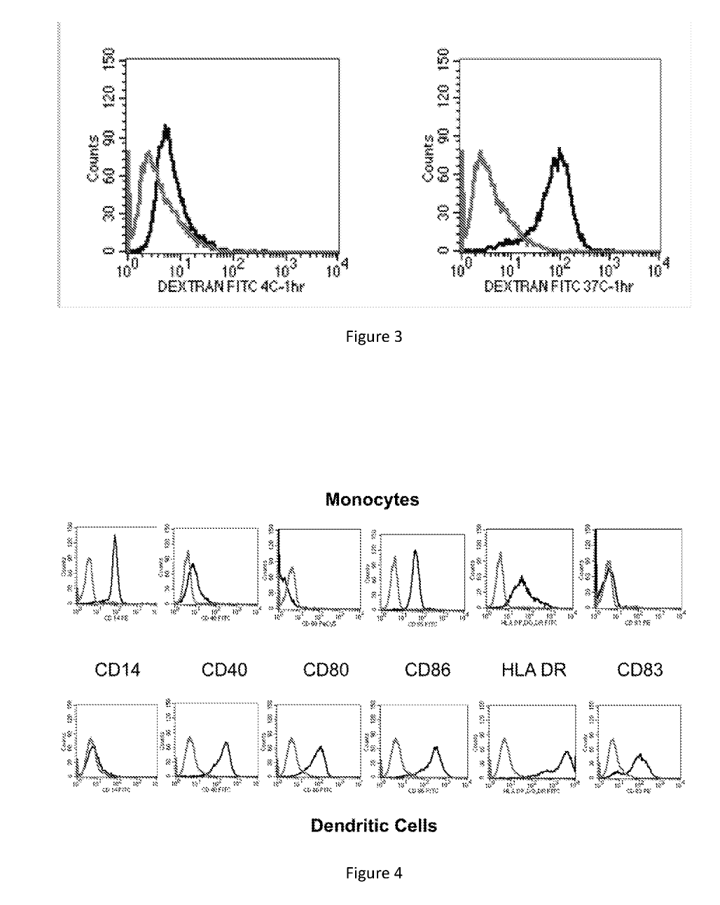

On day 6, immature DCs (1×10⁶ cells/ml) were co-incubated with tumor cell lysate (antigen), β-glucan and sodium hyaluronate (TL-BG-HA) complex for 24-48 hours. Antigen uptake by Dendritic cells was evaluated by flowcytometric analysis using Dextran coated FITC beads added to the composition comprising ex-vivo generated dendritic cells in combination with β-glucan and sodium hyaluronate (DC-TL-BG-HA) (FIG. 3). DC-TL-BG-HA composition was then cryopreserved using standard protocols for cryopreservation of human cells, till further use.

### Example 5

### Phenotypic Analysis of DCs in DC-TL-BG-HA Composition

The composition comprising ex-vivo generated antigen-loaded dendritic cells in combination with β-glucan and sodium hyaluronate (DC-TL-BG-HA) was analyzed based on phenotypic characterization for expression of the surface markers including CD14, CD40, CD80, CD86, HLD DR & CD83 (FIG. 4).

### Example 6

### Functional Validation of DC-TL-BG-HA Composition

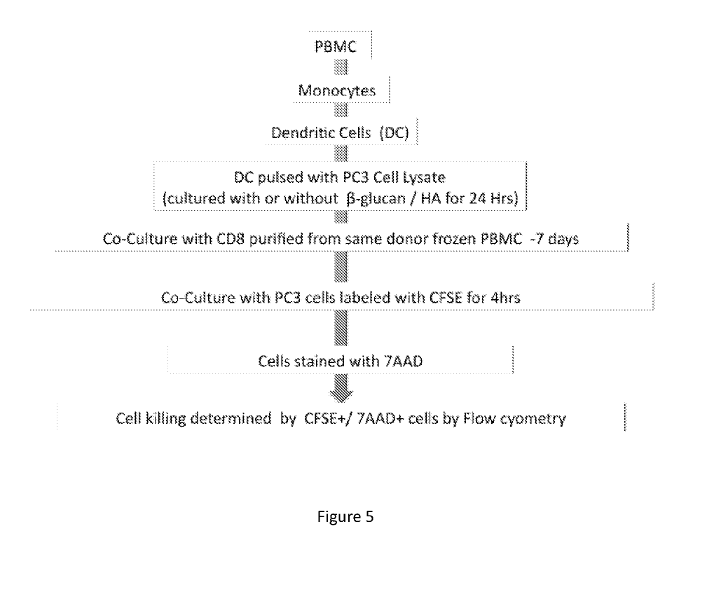

Since functional efficacy of autologus human Dendritic Cells cannot be tested in animal models for evaluating functional efficacy, an ex-vivo experimental model was used as described in the flowchart (FIG. 5).

Dendritic Cells (DCs) were generated from blood sample from an healthy donor. Tumor lysate were prepared from an established Prostate Cancer cell line (PC3) and was complexed with BG-HA, as described above. Experimental groups included

1) CD8 (T cell)—control group

2) Dendritic Cells+CD8 (DC+CD8)

3) Dendritic Cells+Tumor Lysate+CD8 (DC-TL+CD8)

4) Dendritic Cells+Tumor Lysate+sodium hyaluronate+CD8 (DC-TL-HA+CD8)

5) Dendritic Cells+Tumor Lysate+β-glucan+CD8 (DC-TL-BG+CD8)

6) Dendritic Cells+Tumor Lysate+sodium hyaluronate+CD8 (DC-TL-BG-HA+CD8)

Autologus CD8 cells were purified from PBMCs of the same donor (used for DC generation) for 7 days. Following this step, the CD8+ cells were mixed with PC3 tumor cells labeled with a dye (CFSE), for 4 hours. Subsequently, the PC3 cells were stained with another dye—7AAD (stains only dead cells) and cell killing was determined by CFSE+/7AAD+ cells by Flow cytometry. Comparison between various experimental groups, shown in the FIG. 5, confirmed that the DC-TL-BG-HA composition described in this invention provides the most significantly enhanced tumor cell killing ex-vivo, by autologus CD8 cells.

### Example 7

### Clinical Study on Dendritic Cell Vaccine in Chronic Lymphocytic Leukemia (CLL) Patients

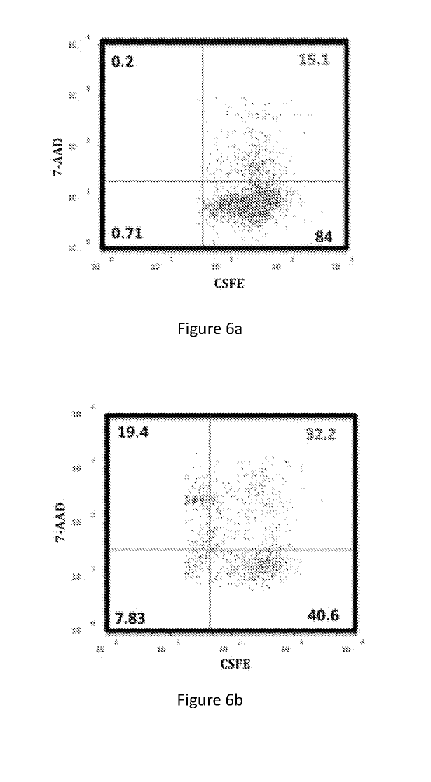

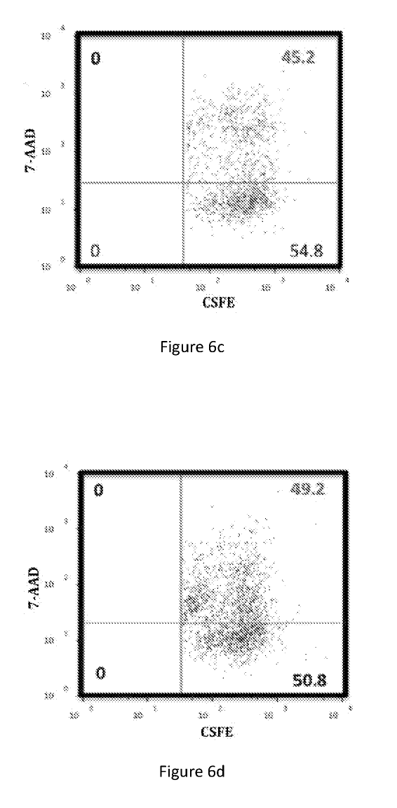

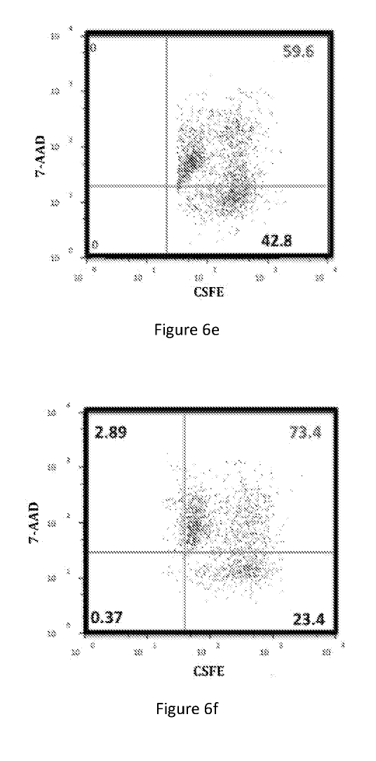

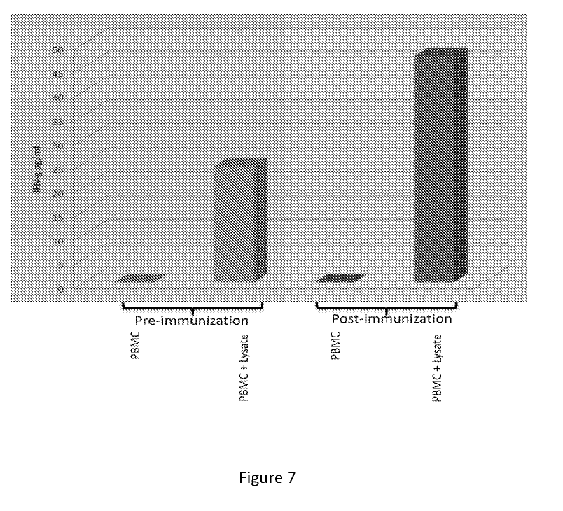

6 patients of chronic lymphocytic leukemia were given 3 intradermal injections, l ml total volume given at multiple sites (100 μl at each site), of the composition comprising ex-vivo generated antigen-loaded dendritic cells in combination with β-glucan and sodium hyaluronate (DC-TL-BG-HA) at 2 weekly intervals. Blood samples taken before and after intradermal injections were analyzed for T and B cell phenotypes and in-vitro production of g-IFN in response to ex-vivo tumor antigen challenge (FIG. 6). Effect of composition (DC-TL-BG-HA) of the present invention on T/B cell population in peripheral blood of some of the chronic lymphocytic leukemia (CLL) patients is presented below in Table 1.

TABLE 1
Effect of composition (DC-TL-BG-HA) of the present invention on T/B cell
population in peripheral blood of CLL patient

| | | CD4 | | CD8 | | CD19 | |
|---|---|---|---|---|---|---|---|
| Patient | Clinical | Preimmu- | Postimmu- | Preimmu- | Postimmu- | Preimmu- | Postimmu- |
| No | ID | nization | nization | nization | nization | nization | nization |
| 1 | 58434 | 3.4 | 0. [symbol] 7 | 2.29 | 3. [symbol] | 72.19 | [symbol] .3 |
| 2 | 48 [symbol] 5 | 5.67 | [symbol] | 3.57 | [symbol] .7 | 89.34 | [symbol] 8.4 |
| 3 | 2211 [symbol] 26 | 6.83 | 8 | 3.71 | [symbol] .7 | 88.42 | [symbol] |
| 4 | 169532 | 1.95 | 1. [symbol] | 1.93 | 3. [symbol] 2 | 94.12 | [symbol] |
| 5 | 11 [symbol] 49 | 8.99 | [symbol] 7 | 12 | 12.97 | 71.84 | 49.4 [symbol] |

[symbol]

indicates data missing or illegible when filed

Results shown above as well as FIG. 6 demonstrate that intradermal injections with the composition comprising ex-vivo generated antigen-loaded dendritic cells in combination with β-glucan and hyaluronic acid (DC-TL-BG-HA) of this invention induces increase in the number of CD8+ T lymphocytes and increased production of g-IFN indicating enhanced antigen-specific T cell response.

The above descriptions and examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

1. A composition comprising ex-vivo generated antigen-loaded dendritic cells in combination with β-glucan and hyaluronic acid or pharmaceutically acceptable derivatives thereof.

2. The composition as claimed in claim 1, wherein the β-glucan or it's pharmaceutically acceptable derivatives has at least one of β (1,3), β (1,6), β (1,3/1,4), and β (1,3/1,6) glycosidic linkages.

3. The composition as claimed in claim 1, wherein the β-glucan is a linear (1, 3) β-glucan.

4. The composition as claimed in claim 1, wherein the β-glucan is micronized or hydrolyzed oligosaccharide or small 10-20 mer polysaccharides or equivalent synthetic oligosaccharides.

5. The composition as claimed in claim 1, wherein the β-glucan has molecular weight of 2000-200,000, preferably in the range of 10,000-20,000 Dalton.

6. The composition as claimed in claim 1, wherein the β-glucan or it's pharmaceutically acceptable derivatives is in the range of 0.1% to 10% on w/v basis.

7. The composition as claimed in claim 1, wherein the β-glucan or it's pharmaceutically acceptable derivatives is in the range of 1% to 5% on w/v basis.

8. The composition as claimed in claim 1, wherein the hyaluronic acid or it's pharmaceutically acceptable derivatives is in the range of 0.1% to 5% on w/v basis, preferably in the range of 0.5% to 2.5% on w/v basis.

9. The composition as claimed in claim 1, wherein the hyaluronic acid or it's pharmaceutically acceptable derivatives is in the range of 0.5%-2% on w/v basis.

10. A pharmaceutical formulation comprising the composition as claimed in claim 1, and optionally pharmaceutically acceptable excipients.

11. The pharmaceutical formulation as claimed in claim 10, wherein the formulation is delivered through injection, biocampitable scaffold or implant by intra-dermal, sub-cutaneous, intra-muscular, intra-tumoral, or intra-nodal administration.

12. A method for preparing a composition comprising ex-vivo generated antigen-loaded dendritic cells in combination with β-glucan and hyaluronic acid comprising of: loading ex-vivo generated dendritic cells with an antigen preparation in presence of a mixture of β-glucan and hyaluronic acid or pharmaceutically acceptable derivatives thereof.

13. Use of the composition as claimed in claim 1 for enhancing antigen-specific T cell response against solid tumor or hematological cancer cells.

14. A method for treatment and/or prevention of cancer progression, recurrence, and/or metastasis in a cancer patient by administering to a patient in the need thereof a therapeutically effective amount of a composition comprising of ex-vivo generated antigen-loaded autologus dendritic cells in combination with β-glucan and hyaluronic acid or pharmaceutically acceptable derivatives thereof.
